(19) United States
(12) Patent Application Publication
(10) Pub. No.: US 2011/0091552 A1
(43) Pub. Date: Apr. 21, 2011

(54) Use of Bcl Inhibitors for the Treatment of Scarring Caused By Cutaneous Wounds, Burns and Abrasions

(76) Inventors: Tim McCaffrey, Silver Spring, MD (US); Dmitry Gagarin, College Park, MD (US); Zhaoqing Yang, Arlington, VA (US)

(21) Appl. No.: 12/978,776

(22) Filed: Dec. 27, 2010

Related U.S. Application Data
(63) Continuation-in-part of application No. 11/848,459, filed on Aug. 31, 2007, now Pat. No. 7,857,804.

(51) Int. Cl. A61K 31/357; A61K 9/14; A61K 31/352; A61K 31/11; A61K 31/137; A61K 31/395; A61K 31/09; A61P 17/00; A61P 17/02

(52) U.S. Cl. 424/484; 514/450; 514/456; 514/700; 514/653; 514/183; 514/716

(57) ABSTRACT

The present invention relates to use of Bcl inhibitors for the prevention of fibroproliferation resulting in the growth of visible or disfiguring scar tissue on human skin, including without limitation keloids and hypertrophic scars. In particular, the present invention relates to the new use of small molecule inhibitors of the Bcl-2/Bcl-XL family of anti-apoptotic proteins for the treatment of cutaneous wounds, burns and abrasions during the healing phase.

### CROSS REFERENCE TO RELATED APPLICATIONS

This application is a Continuation-in-Part of application Ser. No. 11/848,459, filed Mar. 6, 2008, claiming priority under 35 USC 120, the entire contents of which are incorporate herein by reference in its entirety.

### STATEMENT REGARDING FEDERALLY SPONSORED RESEARCH OR DEVELOPMENT

This invention was made, in part, with government support under NIH AG12712 awarded by NIH/National Institutes on Aging. The government has certain rights in the invention.

### FIELD OF THE INVENTION

The present invention relates to use of Bcl inhibitors for the prevention of fibroproliferation resulting in the growth of visible or disfiguring scar tissue on human skin, including keloids and hypertrophic scars. In particular, the present invention relates to the new use of small molecule inhibitors of the Bcl-2/Bcl-XL family of anti-apoptotic proteins for the treatment of cutaneous wounds, burns and abrasions during the healing phase.

### DESCRIPTION OF RELATED ART

The underlying cause of injury-induced fibroproliferation is that the reactive repair cells respond to the injury, but fail to undergo apoptosis when the wound is repaired. Thus, they continue to repair the wound, and thus cause excessive matrix production, and in the process forming scar tissue beyond that necessary to repair the wound. Such fibroproliferation can result in the appearance of hypertrophic scars or keloids, which can be both unsightly and painful.

Thus, the topical application of a composition containing a component capable of slowing or stopping fibroproliferative cellular growth would function as an effective anti-scarring medication. Bcl inhibitors, particularly small molecule inhibitors of the Bcl-2/Bcl-XL family of anti-apoptotic proteins, are known to counteract fibroproliferative cellular growth.

The problem of fiberproliferative cellular growth has been addressed repeatedly in the context of intravascular Drug eluting stents (DES), which are stents with coatings of suppressive agents. The use of intravascular stents is an area of medicine wherein fibroproliferative cellular growth can be particularly dangerous, as extraordinary cell growth triggered by the introduction of a stent can result in partial or total blockage of the blood vessel.

Oncology is another area of medicine in which the use of drugs counteracting fibroproliferative cell growth is well known.

US20050239873A1 discloses 2-methoxy antimycin derivatives or analogs and the methods for treating apoptosis-associated diseases such as neoplastic disease (e.g., cancer) or other proliferative diseases associated with the over-expression of a Bcl-2 family member protein.

US20020137732A1 discloses promotion of apoptosis in tumor cells using tricyclo-dibenzo-diazocine-dioxides, non-peptide pharmaceuticals, which are cell permeable small molecules that bind to a pocket of Bcl-2 and block the Bcl-2 anti-apoptotic function in cancer cells and tumor tissue exhibiting Bcl-2 protein over expression.

US20060247318 A1 discloses the method of inhibiting Stat3 activity in a cell by contacting the cell with “STA-21” or its derivative, analog, prodrug or pharmaceutically acceptable salt and their use for inducing cell death & sensitizing cells. They inhibit the growth of a cell with elevated Stat3 activity thus inducing apoptosis (hyperproliferative disease) and/or cell cycle arrest in a cell. Any oncolytic agent that is routinely used in a cancer therapy is used in the compositions.

US20060247305 discloses the method of inhibiting anti-apoptotic Bcl-2 family members in a cell by contacting the cell with a compound with the following formulae.

Formulae:

The method of rendering further comprises contacting the cell with an inducer of apoptosis (which is chemotherapeutic agent or radiation). Disorder responsive to the induction of apoptosis is a hyperproliferative disease, viz., cancer. By inhibiting anti-apoptotic Bcl-2 family members, compounds disclosed sensitize cells to the inducers of apoptosis and, in some instances, they themselves induce apoptosis.

US20060241067A1 discloses methods for detecting & inhibiting angiogenesis, cell migration, cell adhesion and/or cell survival in endothelial and non-endothelial cells as well as in normal and tumor cells.

US20060178435A1 discloses the preparation and use of apogossypolone that functions as an inhibitor of Bcl-2 family proteins for inhibiting hyperproliferative cell growth, inducing apoptosis in cells and for sensitizing cells to the induction of apoptotic cell death. The present invention includes the method of treating, ameliorating or preventing a disorder responsive to the induction of apoptosis and hyperproliferative disease (cancer) in an animal. This invention also contemplates that the inhibitors of anti-apoptotic Bcl-2 family proteins satisfy an unmet need for the treatment of multiple cancer types, either when administered as monotherapy (by inducing apoptosis) in cancer cells or when administered in a temporal relationship with other cell death-inducing cancer therapeutic drugs or radiation therapies.

US20060111288A1 discloses a method for the treatment of cancer by application of anti-tumor agent HA14-1, which causes intra-mitochondrial generation of reactive oxygen species in tumor cells by a mechanism independent of the peripheral benzodiazepine receptor. This invention also includes a method for inducing apoptosis of cells and for sensitizing cells to anti-neoplastic treatment.

WO02097053 discloses naturally occurring and chemically synthesized small molecules antagonists of Bcl-2 family proteins particularly gossypol derivatives, and their use as antagonists of the anti-apoptotic effects of Bcl-2 and Bcl-XL proteins. A gossypol compound, or an analogue thereof, is administered in conjunction with a tumor cell apoptosis promoting agent (e.g., Geranylgeraniol[3,7,11,15-tetramethyl-2,6,10,14hexadecatraen-1-ol]). The present invention also contemplates that increasing tumor apoptosis reestablishes normal apoptotic control associated with basal levels of Bcl-2 and/or Bcl-XL expression.

US20030199489A1 discloses small molecule antagonists of Bcl-2 family proteins such as Bcl-2 and/or Bcl-XL, in particular, non-peptide cell permeable small molecules (e.g., tricyclo-dibenzo-diazocine-dioxides) that bind to a pocket in Bcl-2/Bcl-XL and block the anti-apoptotic function of these proteins in cancer cells and tumor tissues exhibiting Bcl-2 protein over expression. This patent further discloses that the small molecules of the invention are active at the BH3 binding pocket of Bcl-2 family proteins (e.g., Bcl-2, Bcl-XL, and Mcl-1).

U.S. Pat. No. 5,776,905 discloses, apoptotic regression of neointimal cells due to reduction of Bcl-X levels by treatment of the cells with antisense genes interfering with transcription/translation. The patent further discloses that Bcl-X is elevated in atherosclerotic lesions and claims methods to use the antisense oligonucleotide for the treatment of intimal hyperplasia, restenosis, and atherosclerosis.

U.S. Ser. No. 11/848,459 discloses the use of Bcl inhibitors for the prevention of fibroproliferative reclosure of dilated blood vessels and other iatrogenic fibroproliferative disorders, including the use of small molecule inhibitors of the Bcl-2/Bcl-XL family of anti-apoptotic proteins for the treatment of post-angioplasty restenosis, in-stent restenosis and the like.

The use of topical anti-scarring compositions is also well known. Known compositions address the formation of scar tissue using pH adjustment, antifibrotics and angiogenesis inhibitors.

For example, U.S. Pat. No. 7,323,184 discloses methods and compositions for inhibiting scar formation on wound region. In certain embodiments, the methods include administering an alkalinization agent in combination with hyaluronic acid to a wound site to reduce the formation of scars at the wound site.

U.S. Pat. No. 6,060,474 discloses methods for reducing or preventing scar tissue formation by administering an antifibrotic amount of a fluoroquinolone, and also means for inhibiting the activity of fibroblast cells, including their proliferation, metabolism, and invasion into tissue.

U.S. Pat. No. 6,638,949 discloses a method and compositions for inhibiting excessive scar formation and adhesions by administering to a patient in need thereof an effective amount of an angiogenesis inhibitor, such as a fumagillol derivative like 0-chloroacetylcvarbamoyl-Fumagillol (TNP-470, TAP Pharmaceuticals), thalidomide, or selective drug having more than one activity, such as a minocycline or peniciliamine which also have antibiotic activity.

What is needed is a topical application of small molecule inhibitors of the Bcl-2/Bcl-XL family of anti-apoptic proteins for the treatment of scarring in cutaneous wounds and conditions, where there is the potential for visible scarring due to extraordinary fibroproliferative cellular growth.

### SUMMARY OF THE INVENTION

The present invention relates to the new use of small molecule inhibitors of the Bcl-2/Bcl-X.sub.L family of anti-apoptotic compounds for the treatment of scarring in cutaneous wounds, burns and abrasions, both surgical and non-surgical. The Bcl inhibitory compounds of the present invention are incorporated into a dermatological composition, which is then applied to a cut or wound to prevent the fibroproliferative cellular growth that leads to visible scarring.

The present invention is based on the discovery that cells which are resistant to apoptosis contribute to the fibroproliferative process. The present invention is further based on the discovery that the Bcl-X_{L} gene is increased in cells that are resistant to apoptosis, which is also called as programmed cell death. The small molecules of the present invention block the Bcl family members and convert the cells that are resistant to apoptosis into cells that are sensitive to apoptosis. Furthermore, the present invention is based on the discovery that genetic inhibition by expression of short-hairpin RNA (shRNA) that has the effect of causing targeted degradation of the Bcl-XI mRNA and thus reduced protein synthesis, also cause cells to become more sensitive to apoptosis.

The small molecules of the present invention comprises 2-methoxyantimycin A₃ (abbreviated AA3 in the present application is an antimycin analog which binds the hydrophobic groove of Bcl-2 and Bcl-XI and inhibits their antiapoptotic function); Antimycin A (Chemical composition similar to AA3 above, except containing a mixture of antimycins A1, A2, A3, and A4); 2,9-dimethoxy-11,12-dihydrodibenzo[c,g][1,2]-diazocine 5,6-dioxide (A) and 5,5′-Dimethoxy-2,2′-dinitrosobenzyl (B) Used as a mixture of the tautomers (A) and (B) (abbreviated as BCLI in present application); 2,3-DCPE HCl (2,[[3-(2,3-dichlorophenoxy)propyl]amino]ethanol-HCl); Gossypol (2,2′-bis(8-Formyl-1,6,7-trihydroxy-5-isopropyl-3-methylnaphthal-ene)); HA14-1 (Ethyl-2-amino-6-bromo-4-(1-cyano-2-ethoxy-2-oxoethyl)-4H-chromene-3-carb-oxylate).

In a preferred embodiment, a dermatological composition for topical treatment of a dermatological disorder, comprising water (Aqua) 30-100% wt., at least one small molecule inhibitor of Bcl protein 0.1%-30% wt., at least one dermatologically acceptable carrier 3.0%-10% wt., and wherein weight % is based on the total weight of the composition and the weight percentages of all components in the composition totals 100%.

In another preferred embodiment, the dermatological composition described herein, wherein said composition is an emulsion having an oil phase and an aqueous phase.

In another preferred embodiment, the dermatological composition described herein, further comprising a dermatologically acceptable agents selected from the group consisting of emulsifiers, surfactants, suspending agents, antioxidants, chelates, emollients, humectants, fluid alkyl alcohols, thickening agents, polysiloxanes, modified polysiloxanes, pH modifiers, and mixtures thereof.

In another preferred embodiment, the dermatological composition described herein, wherein said small molecule inhibitor of Bcl protein is present in said aqueous phase or said oil phase.

In another preferred embodiment, the dermatological composition described herein, wherein said composition is in a lotion, cream, emulsion, suspension, ointment, shampoo, gel, aerosol, solution, paste, suspension, skin cleanser, bar, film, bath, soak, spray, stick, powder, or foam, or other pharmaceutically acceptable topical dosage form.

In another preferred embodiment, dermatological composition for topical treatment of a dermatological disorder, comprising water (Aqua) 30-100% wt., one or more small molecule inhibitors of Bcl proteins 0.1%-30% wt., at least one emulsifier 3.0%-10% wt., wherein the dermatological composition is in the form of a cream or an ointment, and wherein weight % is based on the total weight of the composition and the weight percentages of all components in the composition totals 100%.

In another preferred embodiment, the dermatological composition described herein, comprising water (Aqua) 30-97% wt., one or more small molecule inhibitors of Bcl proteins 0.1%-10% wt., at least one emulsifier 3.0%-10% wt., wherein the dermatological composition is in the form of a cream or an ointment, and wherein weight % is based on the total weight of the composition and the weight percentages of all components in the composition totals 100%.

In another preferred embodiment, a dermatological composition for topical treatment of a dermatological disorder, comprising water (Aqua) 30-97% wt., one or more small molecule inhibitors of Bcl proteins 0.1%-5% wt., at least one emulsifier 3.0%-10% wt., wherein the dermatological composition is in the form of a cream or an ointment, and wherein weight % is based on the total weight of the composition and the weight percentages of all components in the composition totals 100%.

In another preferred embodiment, the dermatological composition described herein, wherein the one or more small molecule inhibitors of Bcl proteins is selected from the group comprising 2-methoxyantimycin A3 and its analogs; Antimycin A; 2,9-dimethoxy-11,12-dihydrodibenzo[c,g][1,2]-diazocine 5,6-dioxide (A); 5,5′-Dimethoxy-2,2′-dinitrosobenzyl (B); 2, [[3-(2,3-dichlorophenoxy)propyl]amino]ethanol-HCl; 2,2′-bis(8-Formyl-1,6,7-trihydroxy-5-isopropyl-3-methylnaphthalene); and Ethyl-2-amino-6-bromo-4-(1-cyano-2-ethoxy-2-oxoethyl)-4H-chromene-3-carboxylate, alone or in combination thereof.

In another preferred embodiment, the dermatological composition described herein, further comprising one or more antibiotic ingredients.

In another preferred embodiment, the dermatological composition described herein, further comprising one or more analgesic ingredients.

In another preferred embodiment, the dermatological composition described herein, in the form of a lotion, cream, emulsion, suspension, ointment, shampoo, gel, aerosol, solution, paste, suspension, skin cleanser, bar, film, bath, soak, spray, stick, powder, or foam, or other pharmaceutically acceptable topical dosage form.

In another preferred embodiment, the dermatological composition described herein, in a delivery vehicle selected from a single use, individualized, sterile packet, an aerosol spray, a pump spray, a pre-soaked or infused bandage or dressing, a pre-soaked wipe, an infused film for application to the skin, or an infused sponge with applicator stick for use in oral care to treat mouth sores.

A method of topically treating a dermatological disorder characterized by excessive fibroproliferative growth of cutaneous tissue in a patient having a wound, burn or abrasion, comprising providing a dermatological composition as described herein, and topically applying a quantity of the composition to a portion of the skin of the patient affected by the disorder to treat the excessive fibroproliferative growth.

The method of topically treating a dermatological disorder described herein, wherein the dermatological composition is applied in a resorbable mesh to prevent adhesion and fibrosis when there is excessive scarring after surgical interventions such as surgical adhesions.

The method of topically treating a dermatological disorder described herein, further comprising wherein the dermatological composition is delivered in a hydrogel to effect a slow release of the small molecule BCl inhibitor and prevents the repair of cells from becoming excessively resistant to apoptosis and thereby facilitates normal cutaneous repair.

### BRIEF DESCRIPTION OF THE DRAWINGS

FIG. 1: depicts the effect of Bcl inhibitor on sensitivity to apoptosis.

FIG. 2: depicts the effect of AA, BCLI, interferon gamma (IFN) and SP600125 (SP).

FIG. 3: depicts the effect of 2-Methoxy-antimycin A3(AA3) and Gossypol on the sensitivity to apoptosis in human lesion cells.

FIG. 4: depicts HA14-1 and DCPE in an MTT Assay for apoptosis sensitivity.

### DETAILED DESCRIPTION OF THE INVENTION

### Definitions

The term “apoptosis” refers to a regulated network of biochemical events which lead to a selective form of cell suicide, and is characterized by readily observable morphological and biochemical phenomena, such as the fragmentation of the deoxyribo-nucleic acid (DNA), condensation of the chromatin, which may or may not be associated with endonuclease activity, chromosome migration, margination in cell nuclei, the formation of apoptotic bodies, mitochondrial swelling, widening of the mitochondrial cristae, opening of the mitochondrial permeability transition pores and/or dissipation of the mitochondrial proton gradient and the like.

The term “keloid” refers to a type of scar, which depending on its maturity, is composed of mainly either type III (early) or type I (late) collagen, and often grows beyond the boundaries of the original wound to the skin. It is a result of an overgrowth of granulation tissue (collagen type 3) at the site of a healed skin injury which is then slowly replaced by collagen type 1. Keloids are firm, rubbery lesions or shiny, fibrous nodules, and can vary from pink to flesh-colored or red to dark brown in color.

The term “hypertrophic scar” refers to a cutaneous condition characterized by the formation of excess, raised scar tissue, but not growing beyond the boundary of the original wound. The present invention relates to the new use of small molecule inhibitors of the Bcl-2/Bcl-XL family of anti-apoptotic proteins for the treatment of the formation of scar tissue on human skin. Methods and compositions for treating skin conditions in humans with the potential for scarring, including wounds, ulcers, rashes, burns, abrasions, and other relevant injuries are provided. The invention contemplates the use of an aqueous or emollient medium having one or more Bcl inhibitory compounds to form a dermatological composition.

Certain preferred compounds and uses are described below. The present invention is not limited to these particular compositions and uses.

As Bcl-X and Bcl-2 are an important part of cell survival in human skin, it is envisioned that the drugs would be used locally at the site of cutaneous trauma, or after other physician-initiated (iatrogenic) tissue damage. Local administration can be achieved in a wide variety of ways.

In one embodiment, the composition is delivered to the site of cutaneous damage directly by application of one of the following varieties of composition: a lotion, cream, emulsion, suspension, ointment, gel, paste, film, bath, soak, spray, stick, powder, or foam.

In another embodiment, the chosen composition may be delivered using one of: a single use, individualized, sterile packet, an aerosol spray, a pump spray, a pre-soaked or infused bandage or dressing, a pre-soaked wipe, an infused film for application to the skin.

Preferred compositions include an emulsion having an oil phase and an aqueous phase. Non-limiting examples of specific types of emulsions that can be made according to this process include an oil-in-water emulsion, a water-in-oil emulsion, an oil-in-water-in-oil emulsion, and a water-in-oil-in-water emulsion. The formation of a specific type of emulsion will depend on the specific surfactant system and amount of vegetable oil or vegetable oil derivative used in the process.

Emulsion Oils

The oil phase of the present inventive dermatological compositions may comprise about 3 to about 45% by weight, of the overall weight of the composition, of a vegetable oil or a derivative thereof. In a particularly preferred embodiment, the present inventive compositions contain about 3 to about 20% by weight of the vegetable oil or a derivative thereof. Compositions containing about 5 to about 15% by weight of the vegetable oil or a derivative thereof are especially preferred in this regard.

The use of a vegetable oil rather than a mineral oil permits the present inventive compositions to be readily absorbed by the skin and to not block skin pores, allowing the skin to breathe and resulting in the enhanced evaporation of moisture from the skin. Additionally, the vegetable oils or vegetable oil derivatives included in the present inventive compositions are very similar to human sebum, which is beneficial for skin that does not produce sufficient amounts of sebum. Further, the use of vegetable oils or derivatives thereof may optionally be used to provide dermatological compositions that are not greasy.

The vegetable oil or derivative thereof useful in the present compositions can take the physical form of a fluid, semi-solid, solid, or any other form in which vegetable oils are presently known as available. Preferably, the vegetable oil or derivative thereof is selected from the group consisting of castor oil, sunflower oil, a liquid fraction of karite butter, cottonseed oil, canola oil, corn oil, hydrogenated vegetable oil, peanut oil, sesame oil, soybean oil, palm oil, coconut oil, rapeseed oil, safflower oil, cocoa butter, linseed oil, olive oil, almond oil, avocado oil, citrus seed oil, cohune oil, tallow, oat oil, palm kernel oil, rice bran oil, tucum oil, babassu oil, derivatives thereof, and mixtures thereof. Other vegetable oils or vegetable oil derivatives known in the art as useful in topical dermatological compositions are additionally contemplated as within the scope of the present inventive subject matter.

Dermatologically Acceptable Agents

The present inventive dermatological compositions additionally contain remaining amounts of one or more dermatologically acceptable agents. Preferred, non-limiting examples of dermatologically acceptable agents useful in the present inventive compositions are those selected from the group consisting of emulsifiers, surfactants, suspending agents, antioxidants, chelates, preservatives, emollients, humectants, fluid alkyl alcohols, thickening agents, polysiloxanes, modified polysiloxanes, pH modifiers, and mixtures thereof.

In another preferred embodiment, said active therapeutic agent or its pharmaceutically acceptable free base, salt, ester, or solvate is a skin-conditioning agent. Preferably, the skin-conditioning agent is selected from the group consisting of hydrocarbon oils and waxes, silicones, fatty acid derivatives, cholesterol, cholesterol derivatives, di- and tri-glycerides, vegetable oils, vegetable oil derivatives, and liquid nondigestible oils, or blends of liquid digestible or nondigestible oils with solid polyol polyesters, acetoglyceride esters, alkyl esters, alkenyl esters, lanolin and its derivatives, milk tri-glycerides, wax esters, beeswax derivatives, sterols, phospholipids, and mixtures thereof.

Specific examples of skin conditioning agents also include extracts of kola nut, allantoin, aloe, benzoin compositions, coconut oil, collagen, dimethylsulfoxide, emu oil, linoleic acid compositions, jojoba oil, lavender oil, oligomeric proanthocyanidins, sunflower oil, Vitamin A, C, and E formulations, and zinc compositions.

Topical formulations of Vitamin E have been promoted as an aid to wound healing presumably because they have been shown to inhibit collagen synthesis, decrease fibroblast proliferation and reduce inflammation. Furthermore, vitamin E may improve scar characteristics by its hydrating effects. In one study, the synergistic effects of vitamin E and silicone were shown to be superior to silicone alone in the treatment of keloids.

The present invention also contemplates the use in combination with silicone products. In one preferred embodiment, BCl inhibitor compositions are used in combination with 12% silicone gel preparation. Both, silicone gel and silicone sheeting have been previously demonstrated to reduce scar size and erythema and are encompassed herein. The application of silicone after surgical resection has been shown to help prevent the development of hypertrophic scars and keloids in 75% to 85% of cases. Silicone appears to prevent wound desiccation and inhibition of fibroblast production of collagen and glycosaminoglycans.

In this regard, the additional fluid alkyl alcohol may be used as a solvent for the present compositions in place of water. Likewise, the modified polysiloxanes can be used as a carrier for a gel form of the present compositions.

Emulsion

Emulsions of the present inventive dermatological compositions is preferably formed using at least one emulsifier. Non-limiting examples of suitable emulsifiers useful in the present inventive dermatological compositions include straight or branched chain fatty acids, polyoxyethylene sorbitan fatty acid esters, sorbitan fatty acid esters, propylene glycol stearate, glyceryl stearate, polyethylene glycol, fatty alcohols, polymeric ethylene oxide-propylene oxide block polymers, derivatives thereof, pharmaceutically acceptable salts thereof, and mixtures thereof.

In a particularly preferred embodiment, the emulsifier is selected from the group consisting of laureth-4, glyceryl stearate and polyethylene glycol-100/glyceryl stearate, and mixtures thereof. In yet another preferred embodiment, the emulsifier is present in a phase of said emulsion selected from the group consisting of said oil phase, said aqueous phase, and a combination thereof.

Specific, non-limiting examples of preferred amphoteric surfactants useful in the present inventive dermatological compositions are those selected from the group consisting of sodium 3-dodecyl-aminopropionate, sodium 3-dodecylaminopropane sulfonate, sodium lauroamphoacetate, coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyeth-yl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, and mixtures thereof.

Anionic Surfactants

Similarly, preferred, non-limiting examples of anionic surfactants useful in the present inventive dermatological compositions are those selected from the group consisting of alkyl sulfates, alkyl ethoxylated sulfates, beta-alkyloxy alkane sulfonates, alkyl ether sulfates, alkyl glyceryl ether sulfonates, alkyl ether carboxylates, acyl isethionates, acyl sarcosinates, acyl taurines, succinates, alkali metal, ammonium, or alkanolammonium salts thereof, and mixtures thereof.

Specific, non-limiting examples of preferred anionic surfactants useful in the present inventive dermatological compositions are those selected from the group consisting of ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinnate, disodium lauryl sulfosuccinate, diammonium lauryl sulfosuccinate, tetrasodium N-(1,2-dicarboxyethyl)-N-octadecylsulf-osuccinnate, diamyl ester of sodium sulfosuccinic acid, dihexyl ester of sodium sulfosuccinic acid, dioctyl esters of sodium sulfosuccinic acid, docusate sodium, and mixtures thereof.

Cationic Surfactants

Specific, non-limiting examples of preferred cationic surfactants useful in the present inventive dermatological compositions include those selected from the group consisting of behenyl trimethyl ammonium chloride, bis(acyloxyethyl)hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowedimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picolin benzyl ammonium chloride, polyquarternium, stearalkonium chloride, sterayl dimethylbenzyl ammonium chloride, stearyl trimethyl ammonium chloride, trimethylglycine, and mixtures thereof.

Nonionic Surfactants

Specific, non-limiting examples of preferred nonionic surfactants useful in the present dermatological compositions include those selected from the group consisting of polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethyelen oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and mixtures thereof. A particularly preferred nonionic surfactant useful in the present inventive compositions is laureth-4.

In a particularly preferred embodiment, the present inventive dermatological compositions contain at least two surfactants. In a more particularly preferred embodiment, these surfactants are selected from the group consisting of polyoxyl stearate, laureth-4, and mixtures thereof. In yet another preferred embodiment, the surfactant(s) are present in the oil phase, the aqueous phase, or a combination thereof of the present inventive dermatological compositions.

Suspending Agents

The present inventive dermatological compositions may further contain a suspending agent. Non-limiting examples of suitable suspending agents useful in the present inventive dermatological compositions include alginic acid, bentonite, carbomer, carboxymethylcellulose and salts thereof, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and mixtures thereof.

In a particularly preferred embodiment, the suspending agent is magnesium aluminum silicate. The present inventive compositions may comprise about 0.5 to about 5% by weight of such a preferred suspending agent. In another particularly preferred embodiment, the present inventive compositions may comprise about 1 to about 3.5% by weight of such a preferred suspending agent.

In yet another preferred embodiment, the suspending agent is present in the oil phase, the aqueous phase, or a combination thereof of the present inventive dermatological compositions.

Antioxidants

The present inventive dermatological compositions may further contain an antioxidant. Preferred non-limiting examples of antioxidants useful in the present inventive compositions include those selected from the group consisting of butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and mixtures thereof.

Chelating Agents

The present inventive dermatological compositions may further contain a chelating agent. Preferred non-limiting examples of chelating agents useful in the present inventive compositions include those selected from the group consisting of EDTA, disodium edetate, trans-1,2-diaminocyclohexane-N,N,N′,N′-tetraaceticacid monohydrate, N,N-bis(2-hydroxyethyl)glycine, 1,3-diamino-2-hydroxypropane-N,N,N′,N′-te-traacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′,N′-tetrakis(methylenephosphonic acid), O,O′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, nitrilotripropionic acid, nitrilotris(methylenephosphoric acid), 7,19,30-trioxa-1,4,10,13,16,22,27,33-octaazabicyclo[11,11,11] pentatriacontane hexahydrobromide, triethylenetetramine-N,N,N′,N″,N′″,N′″-hexaacetic acid, pharmaceutically acceptable salts thereof, and mixtures thereof.

Emollients

The present inventive dermatological compositions may further contain an emollient. While these emollients may be present in certain embodiments of the present compositions, that are not critical components of these compositions. Further, when present, these emollients only comprise a small percentage of the present compositions; the compositions do not need to carry high levels of the emollient.

Preferred non-limiting examples of emollients useful in the present inventive compositions include those selected from the group consisting of myristyl lactate, isopropyl palmitate, light liquid paraffin, cetearyl alcohol, lanolin, lanolin derivatives, mineral oil, petrolatum, cetyl esters wax, cholesterol, glycerol, glycerol monostearate, isopropyl myristate, lecithin, and mixtures thereof.

Humectants

The present inventive dermatological compositions may further contain a humectant. Preferred non-limiting examples of humectants useful in the present inventive compositions include glycerin, butylene glycol, propylene glycol, sorbitol, and triacetin.

pH Modifiers

The present inventive dermatological compositions may further contain sufficient amounts of at least one pH modifier to ensure that the composition has a final pH of about 3 to about 11. The preparation of an overall composition having this specific pH in the form of a designated emulsion conveys the unique stability characteristics to the present inventive dermatological compositions.

Preferred non-limiting examples of pH modifiers useful to impart the desired pH to the present inventive compositions are those selected from the group consisting of sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and a mixture thereof. The pH modifiers sodium hydroxide and citric acid are most preferred in this regard.

Additional Active Ingredients

In other embodiments, the composition includes additional active ingredients. Examples of active therapeutic agents or their pharmaceutically acceptable free bases, salts, esters, or solvates useful in the present inventive compositions can be, but are not limited to, those selected from the group consisting of steroids, NSAIDs, antifungal agents, antimicrobials, ureas and salts and derivatives thereof, cancer treating agents, treatment agents for inflammatory disorders, agents intended to protect the skin, modify its appearance, or improve its rate of healing, and mixtures thereof.

Corticosteroids

Preferred corticosteroids useful in the present inventive compositions include, but are not limited to, alclometasone dipropionate, amcinonide, beclamethasone dipropionate, betamethasone benzoate, betamethasone dipropionate, betamethasone valerate, budesonide, clobetasol propionate, clobetasone butyrate, cortisone acetate, desonide, desoximetasone, diflorasone diacetate, diflucortolone valerate, fluclorolone acetonide, flumethasone pivalate, fluocinolone acetonide, fluocinonide, fluocortin butyl, fluocortolone preparations, fluprednidene acetate, flurandrenolide, flurandrenolone, fluticasone propionate, halcinonide, halobetasol propionate, hydrocortisone, hydrocortisone acetate, hydrocortisone butyrate, hydrocortisone propionate, hydrocortisone valerate, methylprednisolone acetate, mometasone furoate, pramoxine hydrochloride, prednisone acetate, prednisone valerate, triamcinolone acetonide, and mixtures thereof.

Antibiotics

In another preferred embodiment, said active therapeutic agent or its pharmaceutically acceptable free base, salt, ester, or solvate is an antimicrobial agent which can include, but is not limited to, those selected from the group consisting of amikacin, bacitracin, colistin, gentamicin, kanamycin, metronidazole, mupirocin, neomycin, netilmicin, polymyxin B, streptomycin, tobramycin, phenols and cresols such as 2,4-dichloro-sym-metaxylenol, parachlorometaxylenol, and parachlorometacresol, bisphenols such as hexachlorophene, dichlorophene, bithionol, triclosan, and fentichlor, salicylanilides such as 4′,5-dibromsalicylanilide, 3′,4′,5-trichlorosalicylanilide, 3′,4′,5-tribromosalicylanilide, and 3,5,dibromo-3′-trifluoromethyl-salicylanilide, carbanilides such as trichlorocarbanilde and 3-trifluoromethyl-4-4′-dichlorocarbanilide, quaternary ammonium compounds such as alkyl-dimethyl benzyl ammonium chloride, alkyl-trimethyl ammonium chloride, alkyl trimethyl ammonium bromide, cetyl-trimethyl ammonium bromide, B-phenoxyethyl-dimethyl-dodecyl ammonium bromide, p-tert-octylphenoxyethoxyethyl-dimethyl-benzyl ammonium chloride, tetradecyl-pyridinium bromide, cetyl pyridinium bromide, cetyl pyridinium chloride, di-(n-octyl)-dimethyl ammonium bromide, alkyl-isoquinolinium bromide, 1-(3-chloroallyl)-3-5,7-triaza-1-azoniaadamantane chloride, and chlorhexidine (1,6,di(N-p-chlorophenylguanidino)hexane), 2-bromo-2-nitropropan-1,3-diol, imidazonidyl urea, ethanol, isopropyl alcohol, and mixtures thereof.

Analgesics

Examples of non-steroidal anti-inflammatory drugs (NSAIDs) include, but are not limited to, Aspirin, Salsalate, Diflunisal, Ibuprofen, Ketoprofen, Nabumetone, Piroxicam, Naproxen, Diclofenac, Indomethacin, Sulindac, Tolmetin, Etodolac, Ketorolac and Oxaprozin.

Antifungals

In another preferred embodiment, said active therapeutic agent or its pharmaceutically acceptable free base, salt, ester, or solvate is an antifungal agent which can include, but is not limited to, those selected from the group consisting of imidazoles, hydroxy pyridones, triazoles, allyl amines, undecylenic acid derivatives, tolnaftate, haloprogin, pyridinethiones, clioquinol, and mixtures thereof.

Preferred antifungal agents useful in the present inventive compositions include, but are not limited to, amphotericin B, butoconazole nitrate, ciclopiroxolamine, clindamycin, clioquinol, clotrimazole, econazole, econazole nitrate, fluconazole, flucytosine, griseofulvin, itraconazole, ketoconazole, miconazole, micronazole, naftifine, nystatin, omadine disulfide, sulconazole, terbinafine, terconazole, tioconazole, tolnaftate, triacetin, undecylenic acid, zinc pyrithione, and mixtures thereof.

Formulation Considerations

The optimal pharmaceutical formulations will be determined by one skilled in the art depending upon considerations such as the particular drug or drug combination and the desired dosage. See, for example, “Remington's Pharmaceutical Sciences”, 18th ed. (1990, Mack Publishing Co., Easton, Pa. 18042) and “Harry's Cosmeticology”, 8th ed. (2000, Chemical Publishing Co., Inc., New York, N.Y. 10016), the entire disclosures of which are hereby incorporated by reference. Such formulations may influence the physical state, stability, rate of in vivo release, and rate of in vivo clearance of the therapeutic agents.

By a “therapeutically effective amount” of a compound of the invention is meant a sufficient amount of the compound to treat the immune disorders delineated herein, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

Dosages

The compositions described herein can, for example, be administered topically with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous administration. Such administration can be used as a chronic or acute therapy. The amount of active ingredient that may be combined with the carrier materials to produce a single dosage form will vary depending upon the host treated and the particular mode of administration.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

Bcl-2 Protein Family Inhibitors

As used herein, a “Bcl-2 protein family inhibitor” refers to a therapeutic compound of any type, including small molecule-, antibody-, antisense-, small interfering RNA-, or microRNA-based compounds, that binds to at least one of Bcl-2, BcI-XL, and Bcl-w, or their corresponding mRNAs including untranslated regions, and antagonizes the activity of the Bcl-2 family related nucleic acid or protein. The inventive methods are useful with any known or hereafter developed Bcl˜2 family inhibitor.

In one preferred embodiment, the Bcl-2 protein family inhibitors includes (Biomol CM-122).

In one preferred embodiment, preferred Bcl-2 protein family inhibitors include: N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide, and N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide, both of which are capable of binding to Bcl-2, BcI-XL, and Bcl-w.

In another preferred embodiment, the BCl inhibitor includes compounds from U.S. Pat. Nos. 7,390,799 and 7,767,684 to Abbott Labs, incorporated herein by reference in their entirety.

Specific Bcl Inhibitors from U.S. Pat. Nos. 7,390,799 and 7,767,684:
N-(4-(4-((2-(4-chlorophenyl)-5,5-dimethyl-1-cyclohex-1-en-1-yl)methyl)pip-erazin-1-yl)benzoyl)-4-(((1R)-3-(morpholin-4-yl)-1-((phenylsulfanyl)methyl-)propyl)amino)-3-((trifluoromethyl)sulfonyl)benzenesulfonamide,3-((chloro(difluoro)methyl)sulfonyl)-N-(4-(4-((2-(4-chlorophenyl)-4,4-dim-ethylcyclohex-1-en-1-yl)methyl)piperazin-1-yl)benzoyl)-4-(((1R)-3-(morphol-in-4-yl)-1-((phenylsulfanyl)methyl)propyl)amino)benzenesulfonamide, 3-((chloro(difluoro)methyl)sulfonyl)-N-(4-(4-((2-(4-chlorophenyl)-1-cyclo-hex-1-en-1-yl)methyl)piperazin-1-yl)benzoyl)-4-(((1R)-3-(morpholin-4-yl)-1-((phenylsulfanyl)methyl)propyl)amino)benzenesulfonamide, N-(4-(4-((2-(4-chlorophenyl)-5,5-dimethylcyclohex-1-en-1-yl)methyl)pipera-zin-1-yl)benzoyl)-4-(((1R)-3-(isopropyl(methyl)amino)-1-((phenylsulfanyl)m-ethyl)propyl)amino)-3-((trifluoromethyl)sulfonyl)benzenesulfonamide, N-(4-(4-((2-(4-chlorophenyl)-1-cyclohepten-1-yl)methyl)piperazin-1-yl)ben-zoyl)-4-(((1R)-3-(isopropyl(methyl)amino)-1-((phenylsulfanyl)methyl)propyl-)amino)-3-((trifluoromethyl)sulfonyl)benzenesulfonamide, 3-((chloro(difluoro)methyl)sulfonyl)-N-(4-(4-((2-(4-chlorophenyl)cyclohex-1-en-1-yl)methyl)piperazin-1-yl)benzoyl)-4-(((1R)-3-((1 S,4S)-2-oxa-5-azab-icyclo[2.2.1]hept-5-yl)-1-((phenylsulfanyl)methyl)propyl)amino)benzenesulf-onamide, N-(4-(4-((2-(4-chlorophenyl)cyclohex-1-en-1-yl)methyl)piperazin-1-yl)benzoyl)-4-(((1R)-3-(morpholin-4-yl)-1-((phenylsulfanyl)methyl)propyl)-amino)-3-((trifluoromethyl)sulfonyl)benzenesulfonamide, 4-(((1R)-3-(7-azabicyclo[2.2.1]hept-7-yl)-1-((phenylsulfanyl)methyl)propy-1)amino)-N-(4-(4-((2-(4-chlorophenyl)cyclohex-1-en-1-yl)methyl)piperazin-1-yl)benzoyl)-3-((trifluoromethyl)sulfonyl)benzenesulfonamide, N-(4-(4-((2-(4-chlorophenyl)cyclohex-1-en-1-yl)methyl)piperazin-1-yl)benz-oyl)-4-(((1R)-3-(2-oxa-5-azabicyclo[2.2.1]hept-5-yl)-1-((phenylsulfanyl)me-thyl)propyl)amino)-3-((trifluoromethyl)sulfonyl)benzenesulfonamide, N-(4-(4-((2-(4-chlorophenyl)-5,5-dimethylcyclohex-1-en-1-yl)methyl)pipera-zin-1-yl)benzoyl)-4-(((1R)-3-(2-oxa-5-azabicyclo[2.2.1]hept-5-yl)-1-((phen-ylsulfanyl)methyl)propyl)amino)-3-((trifluoromethyl)sulfonyl)benzenesulfon-amide, 3-((chloro(difluoro)methyl)sulfonyl)-N-(4-(4-((2-(4-chlorophenyl)-5-,5-dimethylcyclohex-1-en-1-yl)methyl)piperazin-1-yl)benzoyl)-4-(((1R)-3-(2-oxa-5-azabicyclo[2.2.1]hept-5-yl)-1-((phenylsulfanyl)methyl)propyl)amino)-benzenesulfonamide, N-(4-(4-((2-(4-chlorophenyl)cyclohept-1-en-1-yl)methyl)piperazin-1-yl)ben-zoyl)-4-(((1R)-3-(2-oxa-5-azabicyclo[2.2.1]hept-5-yl)-1-((phenylsulfanyl)m-ethyl)propyl)amino)-3-((trifluoromethyl)sulfonyl)benzenesulfonamide, 3-((chloro(difluoro)methyl)sulfonyl)-N-(4-(4-((2-(4-chlorophenyl)cyclohep-t-1-en-1-yl)methyl)piperazin-1-yl)benzoyl)-4-(((1R)-3-(2-oxa-5-azabicyclo[2.2.1]hept-5-yl)-1-((phenylsulfanyl)methyl)propyl)amino)benzenesulfonamide-, N-(4-(4-((2-(4-chlorophenyl)-4,4-dimethylcyclohex-1-en-1-yl)methyl)piper-azin-1-yl)benzoyl)-4-(((1R)-3-(isopropyl(methyl)amino)-1-((phenylsulfanyl)-methyl)propyl)amino)-3-((trifluoromethyl)sulfonyl)benzenesulfonamide, N-(4-(4-((2-(4-chlorophenyl)cyclohex-1-en-1-yl)methyl)piperazin-1-yl)benz-oyl)-4-(((1R)-3-(1,4-oxazepan-4-yl)-1-((phenylsulfanyl)methyl)propyl)amino-)-3-((trifluoromethyl)sulfonyl)benzenesulfonamide, 4-(((1R)-3-(azepan-1-yl)-1-((phenylsulfanyl)methyl)propyl)amino)-N-(4-(4-((2-(4-chlorophenyl)-1-cyclohex-1-en-1-yl)methyl)piperazin-1-yl)benzoyl)-3-((trifluoromethyl)sulfonyl)benzenesulfonamide, N-(4-(4-((2-(4-chlorophenyl)cyclohept-1-en-1-yl)methyl)piperazin-1-yl)ben-zoyl)-4-(((1R)-3-(dimethylamino)-1-((phenylsulfanyl)methyl)propyl)amino)-3-((trifluoromethyl)sulfonyl)benzenesulfonamide, 3-((chloro(difluoro)methyl)sulfonyl)-N-(4-(4-(4-(2-(4-chlorophenyl)cyclohex-1-en-1-yl)methyl)piperazin-1-yl)benzoyl)-4-(((1R)-3-(dimethylamino)-1-((phenylsulfanyl)methyl)propyl)amino)benzenesulfonamide, 3-((chloro(difluoro)methyl)sulfonyl)-N-(4-(4-((2-(4-chlorophenyl)-5,5-dim-ethylcyclohex-1-en-1-yl)methyl)piperazin-1-yl)benzoyl)-4-(((1R)-3-(morphol-in-4-yl)-1-((phenylsulfanyl)methyl)propyl)amino)benzenesulfonamide, N-(4-(4-((2-(4-chlorophenyl)-5,5-dimethyl-1-cyclohex-1-en-1-yl)methyl)piperazin-1-yl)benzoyl)-4-(((1R)-3-(dimethylamino)-1-((phenylsulfanyl)methyl)-propyl)amino)-3-((trifluoromethyl)sulfonyl)benzenesulfonamide, N-(4-(4-((4-(4-chlorophenyl)-5,6-dihydro-2H-pyran-3-yl)methyl)piperazin-1-yl)benzoyl)-4-(((1R)-3-(morpholin-4-yl)-1-((phenylsulfanyl)methyl)propyl)-amino)-3-((trifluoromethyl)sulfonyl)benzenesulfonamide, 3-((chloro(difluoro)methyl)sulfonyl)-N-(4-(4-((2-(4-chlorophenyl)-4,4-dim-ethylcyclohex-1-en-1-yl)methyl)piperazin-1-yl)benzoyl)-4-(((1R)-3-(isopropyl(methyl)amino)-1-((phenylsulfanyl)methyl)propyl)amino)benzenesulfonamide-, 3-((chloro(difluoro)methyl)sulfonyl)-N-(4-(4-((2-(4-chlorophenyl)-5,5-di-methylcyclohex-1-en-1-yl)methyl)piperazin-1-yl)benzoyl)-4-(((1R)-3-(isopropyl(methyl)amino)-1-((phenylsulfanyl)methyl)propyl)amino)benzenesulfonamide-, N-(4-(4-((2-(4-chlorophenyl)-4,4-dimethylcyclohex-1-en-1-yl)methyl)piperazin-1-yl)benzoyl)-4-(((1R)-3-(morpholin-4-yl)-1-((phenylsulfanyl)methyl)-propyl)amino)-3-((trifluoromethyl)sulfonyl)benzenesulfonamide, 3-((chloro(difluoro)methyl)sulfonyl)-N-(4-(4-((2-(4-chlorophenyl)-4,4-dim-ethylcyclohex-1-en-1-yl)methyl)piperazin-1-yl)benzoyl)-4-(((1R)-3-((1S,4S)-2-oxa-5-azabicyclo[2.2.1]hept-5-yl)-1-((phenylsulfanyl)methyl)propyl)amino)benzenesulfonamide, N-(4-(4-((2-(4-chlorophenyl)cyclohex-1-en-1-yl)methyl)piperazin-1-yl)benzoyl)-4-(((1R)-3-(dimethylamino)-1-((phenylsulfanyl)methyl)propyl)amino)-3-((trifluoromethyl)sulfonyl)benzenesulfonamide, N-(4-(4-((2-(4-chlorophenyl)cyclohex-1-en-1-yl)methyl)piperazin-1-yl)benz-oyl)-4-(((1R)-1-((phenylsulfanyl)methyl)-3-(pyrrolidin-1-yl)propyl)amino)-3-((trifluoromethyl)sulfonyl)benzenesulfonamide, 3-((chloro(difluoro)methyl)sulfonyl)-N-(4-(4-((2-(4-chlorophenyl)-4,4-dim-ethylcyclohex-1-en-1-yl)methyl)piperazin-1-yl)benzoyl)-4-(((1R)-3-(dimethyl-amino)-1-((phenylsulfanyl)methyl)propyl)amino)benzenesulfonamide, N-(4-(4-((2-(4-chlorophenyl)cyclohept-1-en-1-yl)methyl)piperazin-1-yl)ben-zoyl)-4-(((1R)-1-((phenylsulfanyl)methyl)-3-(pyrrolidin-1-yl)propyl)amino)-3-((trifluoromethyl)sulfonyl)benzenesulfonamide, 3-((chloro(difluoro)methyl)sulfonyl)-N-(4-(4-((2-(4-chlorophenyl)-5,5-dim-ethylcyclohex-1-en-1-yl)methyl)piperazin-1-yl)benzoyl)-4-(((1R)-1-((phenyl-sulfanyl)methyl)-3-(pyrrolidin-1-yl)propyl)amino)benzenesulfonamide, 3-((chloro(difluoro)methyl)sulfonyl)-N-(4-(4-((2-(4-chlorophenyl)-4,4-dim-ethylcyclohex-1-en-1-yl)methyl)piperazin-1-yl)benzoyl)-4-(((1R)-1-((phenyl-sulfanyl)methyl)-3-(pyrrolidin-1-yl)propyl)amino)benzenesulfonamide, 3-((chloro(difluoro)methyl)sulfonyl)-N-(4-(4-((2-(4-chlorophenyl)cyclohep-t-1-en-1-yl)methyl)piperazin-1-yl)benzoyl)-4-(((1R)-1-((phenylsulfanyl)methyl)-3-(pyrrolidin-1-yl)propyl)amino)benzenesulfonamide, N-(4-(4-((2-(4-chlorophenyl)cyclohex-1-en-1-yl)methyl)piperazin-1-yl)benz-oyl)-4-(((1R)-3-(isopropyl(methyl)amino)-1-((phenylsulfanyl)methyl)propyl)-amino)-3-((trifluoromethyl)sulfonyl)benzenesulfonamide, N-(4-(4-((2-(4-chlorophenyl)-4,4-dimethylcyclohex-1-en-1-yl)methyl)pipera-zin-1-yl)benzoyl)-4-(((1R)-1-((phenylsulfanyl)methyl)-3-(pyrrolidin-1-yl)propyl)amino)-3-((trifluoromethyl)sulfonyl)benzenesulfonamide, 3-((chloro(difluoro)methyl)sulfonyl)-N-(4-(4-((2-(4-chlorophenyl)cyclohex-1-en-1-yl)methyl)piperazin-1-yl)benzoyl)-4-(((1R)-1-((phenylsulfanyl)meth-yl)-3-(pyrrolidin-1-yl)propyl)amino)benzenesulfonamide, N-(4-(4-((2-(4-chlorophenyl)-4,4-dimethylcyclohex-1-en-1-yl)methyl)pipera-zin-1-yl)benzoyl)-4-(((1R)-3-(dimethylamino)-1-((phenylsulfanyl)methyl)propyl)amino)-3-((trifluoromethyl)sulfonyl)benzenesulfonamide, N-(4-(4-((2-(4-chlorophenyl)-4,4-dimethylcyclohex-1-en-1-yl)methyl)pipera-zin-1-yl)benzoyl)-4-(((1R)-3-((1S,4S)-2-oxa-5-azabicyclo[2.2.1]hept-5-yl)-1-((phenylsulfanyl)methyl)propyl)amino)-3-((trifluoromethyl)sulfonyl)benzenesulfonamide N-(4-(4-((4′-chloro(1,1′-biphenyl)-2-yl)methyl)-1-piperazinyl)benzoyl)-4-(((1R)-3-(dimethylamino)-1-((phenylsulfanyl)methyl)propyl)amino)-3-((trifluoromethyl)sulfonyl)benzenesulfonamide or N-(4-(4-((4′-chloro(1,1′-biphenyl)-2-yl)methyl)-1-piperazinyl)benzoyl)-4-(((1R)-3-(4-morpholinyl)-1-((phenylsulfanyl)methyl)propyl)amino)-3-((trifl-uoromethyl)sulfonyl)benzenesulfonamide, N-(4-(4-((2-(4-chlorophenyl)-5,5-dimethyl-1-cyclohex-1-en-1-yl)piperazin-1-yl)benzoyl)-4-(((1R)-3-(morpholin-4-yl)-1-((phenylsulfanyl)methyl)propyl)-amino)-3-((trifluoromethyl)sulfonyl)benzenesulfonamide, or a therapeutically acceptable salt thereof. Further, according to another embodiment, a major application is in the prevention of excessive cutaneous wound repair, which is called hypertrophic scars, or keloids. This embodiment applies to both natural wounds or abrasions, or surgical incisions. The composition is applied topically after injury or surgery, or eluted from a polymeric bandage, suture, or coating to prevent excessive scarring in susceptible individuals.

In another preferred embodiment, the small molecules of the present invention comprises:

2-methoxyantimycin A.sub.3 (Biomol, Plymouth Meeting, Pa.). Molecular formula: C₂₇H₃₈N₂O₉ (abbreviated AA3 in the present application). AA3 is an antimycin analog which binds the hydrophobic groove of Bcl-2 and Bcl-XI and inhibits their antiapoptotic function [1].

Antimycin A (Sigma, A8674, abbrev. AA). Chemical composition similar to AA3 above, except containing a mixture of antimycins A1, A2, A3, and A4.

2,9-dimethoxy-11,12-dihydrodibenzo[c,g][1,2]-diazocine 5,6-dioxide (A) and 5,5′-Dimethoxy-2,2′-dinitrosobenzyl (B). Used as a mixture of the tautomers (A) and (B). (abbreviated as BCLI in present application).

2,3-DCPE HCl (Biomol # AP-306). F.W. 300.6, C.₁₁H₁₅CL₂NO₂—HCl, 2,[[3-(2,3-dichlorophenoxy)propyl]amino]ethanol-HCl.

Gossypol. (Sigma Cat # G8761, FW 518.55), C₃₀H₃₀O₈, 2,2′-bis(8-Formyl-1,6,7-trihydroxy-5-isopropyl-3-methylnaphthalene).

HA14-1 (C₁₇H₁₇BrN₂O₅) F.W. 409.2 (Biomol CM-122) Ethyl-2-amino-6-bromo-4-(1-cyano-2-ethoxy-2-oxoethyl)-4H-chromene-3-carboxylate.

Identification of sensitive and resistant cells: Using cells derived from human atherosclerotic lesions, we identified their apoptotic (programmed cell death) response to a known trigger: fas ligation. According to the above theory, cells which undergo apoptosis in response to fas ligation are ‘normal’ repair cells which would not cause excessive repair/fibrosis. Conversely, cells which are resistant to fas ligation, are proposed to be ‘abnormal’ repair cells which would cause excessive wound repair and hence, scarring.

Microarray analysis of sensitive and resistant cells: The full mRNA expression pattern of the sensitive and resistant cells was examined by Affymetrix microarrays, and genes which were differentially expressed were identified. These genes were further filtered by preference for genes which might alter sensitivity to apoptosis. A set of genes were identified and further examined by quantitative PCR and Western blot. Likely candidate genes were caspase 1, BAD, cyclin D1, STAT6 and Bcl-XI. These results are described in [2]. While the expression patterns suggest that these factors are involved, the proof is whether altering their expression has the predicted effect upon the response to fas ligation.

Immortalization/cloning of sensitive/resistant cells: A problem in working with primary cells is that cells which are initially sensitive to apoptosis become resistant to apoptosis after a short time in culture. Thus, it was desirable to stabilize the cells and so some of these repair cells spontaneously immortalized and then individual cells were plated in separate wells and allowed to grow into separate clonal lines. Then, their sensitivity or resistance to fas ligation was determined. Clones that were sensitive and others that were resistant were selected for further study.

Inhibition and over expression of selected apoptosis regulators: Both inhibitors of the candidate gene products, and genetic over expression of the gene was employed in order to determine whether the gene is actually involved in resistance to apoptosis. Our studies indicated that cyclin D1 inhibitors seem to reverse the resistance to apoptosis [3], though there are prior publications and patents to this effect. A separate manuscript describes the fact that several other genes, while promising targets, did not regulate sensitivity/resistance to apoptosis. The examples include BAD, caspase 1, and STAT6. However, Bcl-XI (BCL2L1) proved to be a very potent modulator of sensitivity to apoptosis [4]. The evidence for this is three-fold:

(1) Genetic over expression of Bcl-XI in cells sensitive to apoptosis converted them to apoptosis-resistant.

(2) Small molecule inhibitors of Bcl-2 and/or Bcl-XI convert cells that are resistant into cells that are sensitive to apoptosis.

(3) Genetic inhibition by expression of short-hairpin RNA (shRNA), which has the effect of causing targeted degradation of the Bcl-XI mRNA and thus reduced protein synthesis, also caused cells to become more sensitive to apoptosis.

In the present examples, AA was used in concentrations of 5-50 μg/ml in Medium 199 with 1% fetal bovine serum (FBS). BCLI was used in concentrations ranging from 5-100 μM. AA3 was used in concentrations from 10-50 μg/ml. Gossypol was used in concentrations from 0.5-5 μM.

Cells for study were derived from human carotid artery atherosclerotic lesions, by published methods [2]. Additionally, cell lines were subcloned and tested for sensitivity to fas ligation. In the present studies, clonal lines with known resistance to fas ligation were used [2].

Induction of apoptosis was achieved by reduction of serum concentration from 10% to 1% FBS for 24 hours prior to adding an activating antibody to fas (CH11, Upstate Biotechnology) at a typical concentration of 50-500 ng/ml. Typically 24-48 hours after fas ligation by CH11, cell survival was determined by measuring metabolic activity by the MTT method. MTT is converted to blue dye by metabolically active and viable cells, and thus cells that have undergone apoptosis do not metabolize MTT, thereby creating less blue color [2].

### EXAMPLES OF STUDIES

### Study Example 1

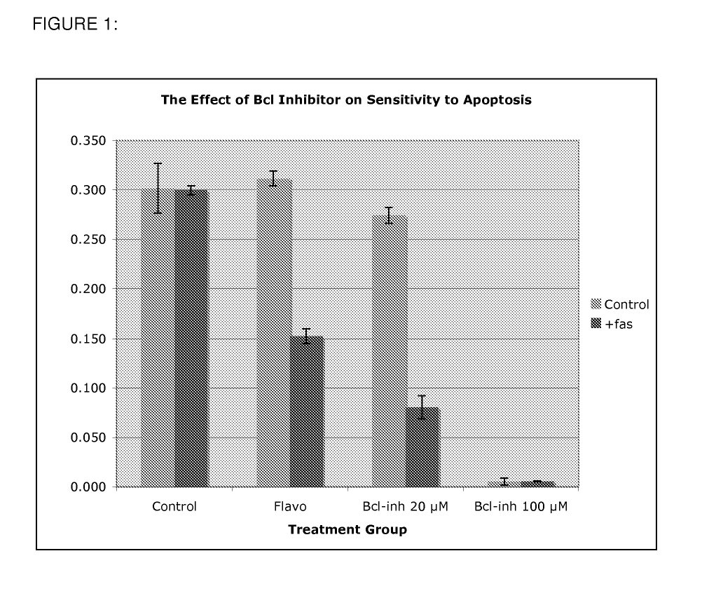

Using the cell line R1, it was observed that pretreatment with BCL-inhibitor (BCLI) at 20 μM had little or no effect upon cell survival in the absence of the activating antibody to fas ligation (CH11, FIG. 1). However, in the presence of fas, the cells pretreated with BCLI underwent a high degree of apoptosis (70% cell death). It is important to reiterate that at this dose (20 μM), BCLI has little or no adverse effect on cell number or metabolism, as demonstrated in the control group which contains the drug, but not a fas ligating agent. Thus, the drug is neither toxic nor capable of inducing apoptosis alone at this concentration.

This compares favorably with a known sensitizing agent, flavopiridol, which has likewise been shown to inhibit restenosis in animal models [5]. At a higher concentration of BCLI (100 μM), there was a substantial effect on cell survival even in the absence of fas ligation. This suggests that at relatively low doses BCLI sensitizes cell to apoptosis induced by fas ligation, while not itself inducing apoptosis. At higher doses, apoptosis is induced by BCLI alone.

Referring to FIG. 1, A human cell line derived from carotid artery atherosclerotic lesion was plated at known density and then subgroups of 3 wells were treated with various drugs prior to exposure to fas ligation by the activating CH₁₁ antibody (+fas). Pretreatment with flavopiridol (Flavo, 50 nM) or BCLI (Bcl-inh, 20 μM or 100 μM) was followed 24 hours later by addition of CH11 activating antibody to fas (50 ng/ml). After fas ligation, cell survival was evaluated 24 hours later by measuring the number of viable cells using the metabolic dye MTT, which is converted to a blue dye that absorbs at 570 nm. Values reflect the mean±s.e.m. (n=3 per group).

### Study Example 2

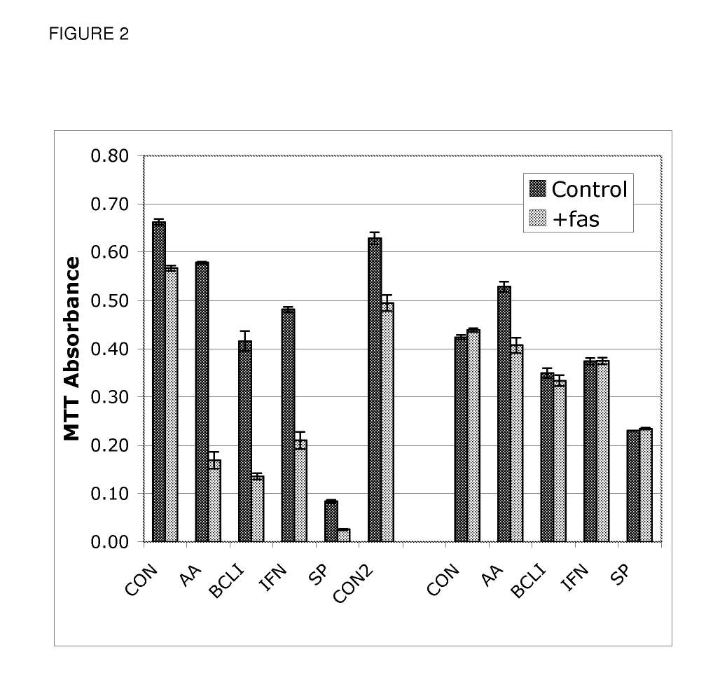

In addition to the BCL-inhibitor, this sensitizing effect was also observed with AA. As above, a human atherosclerosis-derived cell line with known resistance to fas ligation was used for study. In this case, the cell line was stably infected with a retrovirus expressing either an irrelevant marker gene (green fluorescent protein, GFP) or the marker gene and Bcl-XI. In the GFP-only expressing cell line (R1GFP), fas ligation (50 ng/ml CH11) in low serum (1% FBS) led to only 15% cell death (85% survival) (see FIG. 2). However, 24 hour pretreatment with AA3 (20 μg/ml) had only a small effect on cell survival in the absence of fas, but caused marked apoptosis in the presence of fas (70% apoptosis). BCLI (20 μM) had a similar effect, though caused slightly more apoptosis alone. A comparable effect was observed with interferon-gamma (50 U/ml), which is a known sensitizing agent [6]. A MAPkinase inhibitor SP600125 (SP), had a more general inhibitory effect at the selected concentration (20 μM).

In one example, to determine whether the effect of BCLI and AA was via interaction with their intended targets (Bcl-XI and Bcl-2), the drugs were tested in R1 cells over-expressing Bcl-XI (R1 bclXL). In these cells, the BCL inhibitors were markedly less effective, presumably because they had to compete with, and inactivate a much larger amount of endogenous Bcl-XI (see FIG. 2, right bars). Likewise, interferon was less effective, possibly because its effect also depends upon modulating the effect of Bcl-XI. The SP was still an effective inhibitor, though less potent than in control GFP-expressing cells.

Referring to FIG. 2, human atherosclerosis-derived cells were pretreated with the specified drugs for 24 prior to treatment with the activating fas antibody CH11 (50 ng/ml). After the fas treatment, 24 hours later cell survival was measured by MTT wherein dead cells do not generate MTT absorbance. The drugs used were AA (20 μg/ml), BCLI (20 μM), interferon-gamma (IFN, 50 U/ml), and SP600125 (SP, 20 μM). The upper panel reports the MTT optical density (absorbance at 570 nm) without (control) or with fas ligation (+fas) as the mean, with error bars indicating standard error of measurement (s.e.m., n=3 per group). The lower panel expresses % survival based on the MTT values above (MTT+fas/MTT Control)×100.

### Study Example 3

In a format similar to the procedures above, 2 other potential inhibitors were tested: antimycin A3 and gossypol. Antimycin A3 (2-methoxyantimycin A3) is an analog of antimycin which inhibits Bcl-2 and Bcl-XI by binding to their hydrophobic groove. AA3, unlike antimycin A, does not cause general mitochondrial toxicity by inhibiting mitochondrial electron transport.

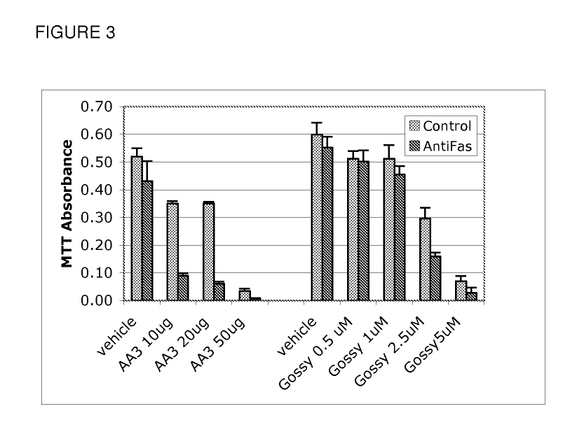

Gossypol is a potential male infertility agent that also counteracts the activity of Bcl family members. As shown in FIG. 3, AA3 is effective at increasing sensitivity to apoptosis from 20% to 80% death at both 10 and 20·mu·g/ml. At higher concentrations, AA3 induced death even in the absence of fas ligation. Gossypol showed a narrow window in which it induced any sensitivity to fas ligation: at 2.5·mu·M it reduced survival in the presence of fas from 80% to 50%, though it also had a significant fas-independent effect on survival at 2.5-5·mu·M.

FIG. 3 depicts the effect of 2-methoxy-antimycin A3 (AA3) and gossypol on the sensitivity to apoptosis in human lesion cells. Human lesion cells were plated and treated as above for 24 hours with the specified concentrations of drug. Fas ligation was initiated with 50 ng/ml of activating antibody CH11 and 24 hours later the number of cell surviving was measured by their metabolism of MTT. Bars are mean±s.d. (n=4 per group). A reduction in MTT levels indicates cell death.

### Study Example 4

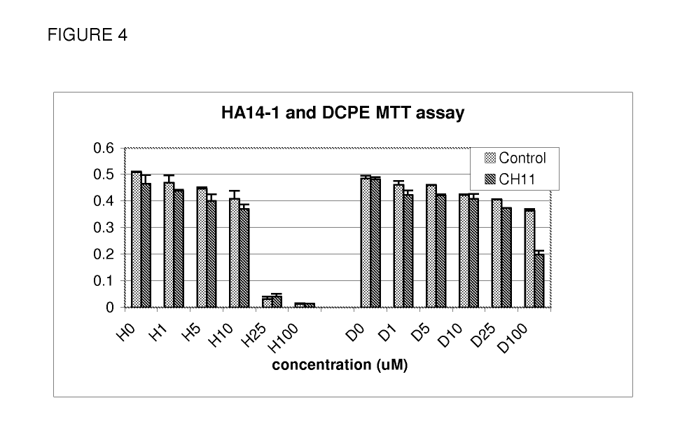

Two other putative Bcl family inhibitors are HA14-1 and 2,3 DCPE. HA14-1 is thought to be a Bcl-2 ligand [7], and 2,3-DCPE is thought to reduce the expression of Bcl-XI and induce apoptosis in tumor cell lines [8]. As shown in FIG. 4, HA14-1 did not demonstrate any ability to sensitize cell to apoptosis, though at concentrations of 25-100 μM, HA14-1 had a fas-independent death-inducing effect on lesion cells. DCPE demonstrated a significant fas-dependent sensitization to apoptosis, but only at the highest tested dose of 100 μM.

FIG. 4 depicts the effect of HA14-1 and 2,3-DCPE on sensitivity to fas-induced apoptosis. Human lesion cells were plated and treated as above for 24 hours with the specified concentrations (1M) of HA14-1 (H) or 2,3-DCPE (D). Fas ligation was initiated with 50 ng/ml of activating antibody CH11 and 24 hours later the number of cell surviving was measured by their metabolism of MTT. Bars are mean±s.d. (n=2 per group). A reduction in MTT levels indicates cell death.

### EXAMPLES OF COMPOSITIONS

References to “Bcl inhibitor” in the below examples include by reference any of the small molecules described beginning in paragraph [57] herein, without limitation.

### Composition Example 1

Water (Aqua) 30-100% wt., Mineral Oil 10-30% wt., Petrolatum 3-10% wt., Sorbitol 3-10% wt., Ceresine Wax 3-10% wt., Sorbitan Sesquioleate 3-10% wt., Lanolin Alcohol 1-3% wt., Bcl inhibitor 1-10% wt., Phenoxyethanol <1%, Ethylhexylglycerin <1%.

### Composition Example 2

Water (Aqua) 30-100% wt., Mineral Oil 10-30% wt., Petrolatum 3-10% wt., Sorbitol 3-10% wt., Ceresine Wax 3-10% wt., Sorbitan Sesquioleate 3-10% wt., Lanolin Alcohol 1-3% wt., Bcl inhibitor 1-7% wt., BHT <1% wt., Phenoxyethanol <1%, Ethylhexylglycerin <1%.

### Composition Example 3

Water (Aqua) 30-100% wt., Mineral Oil 10-30% wt., Petrolatum 3-10% wt., Sorbitol 3-10% wt., Ceresine Wax 3-10% wt., Sorbitan Sesquioleate 3-10% wt., Lanolin Alcohol 1-3% wt., Bcl inhibitor 1-3% wt., BHT <1% wt., Phenoxyethanol <1%, Ethylhexylglycerin <1%.

### Composition Example 4

Water (Aqua) 30-100% wt., Mineral Oil 10-30% wt., Petrolatum 3-10% wt., Sorbitol 3-10% wt., Ceresine Wax 3-10% wt., Sorbitan Sesquioleate 3-10% wt., Lanolin Alcohol 1-3% wt., Bcl inhibitor 1-7% wt., neomycin sulfate/polymyxin B/bacitracin zinc 1-3% wt., BHT <1% wt., Phenoxyethanol <1%, Ethylhexylglycerin <1%.

### Composition Example 5

Water (Aqua) 30-100% wt., Mineral Oil 10-30% wt., Petrolatum 3-10% wt., Sorbitol 3-10% wt., Ceresine Wax 3-10% wt., Sorbitan Sesquioleate 3-10% wt., Lanolin Alcohol 1-3% wt., Bcl inhibitor 1-7% wt., pramoxine/capzasin-P/menthacin/zostrix 1-3% wt., BHT <1% wt., Phenoxyethanol <1%, Ethylhexylglycerin <1%.

### Composition Example 6

Water (Aqua) 30-100% wt., Mineral Oil 10-30% wt., Petrolatum 3-10% wt., Sorbitol 3-10% wt., Ceresine Wax 3-10% wt., Sorbitan Sesquioleate 3-10% wt., Lanolin Alcohol 1-3% wt., Bcl inhibitor 1-7% wt., neomycin sulfate/polymyxin B/bacitracin zinc 1-3% wt., pramoxine/capzasin-P/menthacin/zostrix 1-3% wt., BHT <1% wt., Phenoxyethanol <1%, Ethylhexylglycerin <1%.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

1. A dermatological composition for topical treatment of a dermatological disorder, comprising water (Aqua) 30-100% wt., at least one small molecule inhibitor of Bcl protein 0.1%-30% wt., at least one dermatologically acceptable carrier 3.0%-10% wt., and wherein weight % is based on the total weight of the composition and the weight percentages of all components in the composition totals 100%.

2. The dermatological composition of claim 1, wherein said composition is an emulsion having an oil phase and an aqueous phase.

3. The dermatological composition of claim 2, further comprising a dermatologically acceptable agents selected from the group consisting of emulsifiers, surfactants, suspending agents, antioxidants, chelates, emollients, humectants, fluid alkyl alcohols, thickening agents, polysiloxanes, modified polysiloxanes, pH modifiers, and mixtures thereof.

4. The dermatological composition of claim 2, wherein said small molecule inhibitor of Bcl protein is present in said aqueous phase or said oil phase.

5. The dermatological composition of claim 1, wherein said composition is in a lotion, cream, emulsion, suspension, ointment, shampoo, gel, aerosol, solution, paste, suspension, skin cleanser, bar, film, bath, soak, spray, stick, powder, or foam, or other pharmaceutically acceptable topical dosage form.

6. A dermatological composition for topical treatment of a dermatological disorder, comprising water (Aqua) 30-100% wt., one or more small molecule inhibitors of Bcl proteins 0.1%-30% wt., at least one emulsifier 3.0%-10% wt., wherein the dermatological composition is in the form of a cream or an ointment, and wherein weight % is based on the total weight of the composition and the weight percentages of all components in the composition totals 100%.

7. The dermatological composition of claim 6, comprising water (Aqua) 30-97% wt., one or more small molecule inhibitors of Bcl proteins 0.1%-10% wt., at least one emulsifier 3.0%-10% wt., wherein the dermatological composition is in the form of a cream or an ointment, and wherein weight % is based on the total weight of the composition and the weight percentages of all components in the composition totals 100%.

8. A dermatological composition for topical treatment of a dermatological disorder, comprising water (Aqua) 30-97% wt., one or more small molecule inhibitors of Bcl proteins 0.1%-5% wt., at least one emulsifier 3.0%-10% wt., wherein the dermatological composition is in the form of a cream or an ointment, and wherein weight % is based on the total weight of the composition and the weight percentages of all components in the composition totals 100%.

9. The dermatological composition according to any of claim 1, 6, 7, or 8, wherein the one or more small molecule inhibitors of Bcl proteins is selected from the group comprising 2-methoxyantimycin A3 and its analogs; Antimycin A; 2,9-dimethoxy-11,12-dihydrodibenzo[c,g][1,2]-diazocine 5,6-dioxide (A); 5,5′-Dimethoxy-2,2′-dinitrosobenzyl (B); 2,[[3-(2,3-dichlorophenoxy)propyl]amino]ethanol-HCl; 2,2′-bis(8-Formyl-1,6,7-trihydroxy-5-isopropyl-3-methylnaphthalene); and Ethyl-2-amino-6-bromo-4-(1-cyano-2-ethoxy-2-oxoethyl)-4H-chromene-3-carboxylate, alone or in combination thereof.

10. The dermatological composition according to any of claim 1, 6, 7, or 8, further comprising one or more antibiotic ingredients.

11. The dermatological composition according to any of claim 1, 6, 7, or 8, further comprising one or more analgesic ingredients.

12. The dermatological composition according to any of claim 1, 6, 7, or 8, in the form of a lotion, cream, emulsion, suspension, ointment, shampoo, gel, aerosol, solution, paste, suspension, skin cleanser, bar, film, bath, soak, spray, stick, powder, or foam, or other pharmaceutically acceptable topical dosage form.

13. The dermatological composition according to any of claim 1, 6, 7, or 8, in a delivery vehicle selected from a single use, individualized, sterile packet, an aerosol spray, a pump spray, a pre-soaked or infused bandage or dressing, a pre-soaked wipe, an infused film for application to the skin, or an infused sponge with applicator stick for use in oral care to treat mouth sores.

14. A method of topically treating a dermatological disorder characterized by excessive fibroproliferative growth of cutaneous tissue in a patient having a wound, burn or abrasion, comprising providing a dermatological composition according to any of claim 1, 6, 7, or 8, and topically applying a quantity of the composition to a portion of the skin of the patient affected by the disorder to treat the excessive fibroproliferative growth.

15. The method of claim 14, wherein the dermatological composition is applied in a resorbable mesh to prevent adhesion and fibrosis when there is excessive scarring after surgical interventions such as surgical adhesions.

16. The method of claim 14, further comprising wherein the dermatological composition is delivered in a hydrogel to effect a slow release of the small molecule BCl inhibitor and prevents the repair of cells from becoming excessively resistant to apoptosis and thereby facilitates normal cutaneous repair.
